(19) United States
(12) Patent Application Publication
(10) Pub. No.: US 2022/0165434 A1
(43) Pub. Date: May 26, 2022

(54) CARE PLAN DELIVERY AND ADHERENCE

(71) Applicant: CAREVISOR, INC., Mendham, NJ (US)

(72) Inventors: Cuong V. DO, Coral Gables, FL (US); Tejash SHAH, New Canaan, CT (US)

(21) Appl. No.: 17/602,341

(22) PCT Filed: Apr. 8, 2020
(86) PCT No.: PCT/US20/27258

§ 371 Date: Oct. 8, 2021

Related U.S. Application Data
(60) Provisional application No. 62/832,526, filed on Apr. 11, 2019.
(60) Provisional application No. 62/944,216, filed on Dec. 5, 2019.

(51) Int. Cl. G16H 70/20; G16H 40/20; G16H 40/67; G16H 50/20; G16H 80/00; G16H 20/10; G16H 20/30; G16H 20/60

(52) U.S. Cl. CPC G16H 70/20; G16H 40/20; G16H 40/67; G16H 20/60; G16H 80/00; G16H 20/10; G16H 20/30; G16H 50/20

(57) ABSTRACT

A health care system's compliance engine uses a protocol management system to archive clinical treatment protocols for various clinical conditions in a single standard clinical protocol format, the format including elements such as parameters to be tracked, actions to be taken, symptom to watch for, and a period for each element. Archived protocols include standard protocols and protocols tailored for individual patients. The compliance engine further includes an escalation control system that assesses compliance and suggests remedial action according to plural factors including the clinical protocols, severity of non-compliance, rules regarding patient and caregiver contacts, compliance history of the patient, compliance history of a patient cohort, and machine learning as to probable best escalation paths for optimum outcomes.

### BACKGROUND

This disclosure pertains to care plan information systems including care provider and patient interfaces.

### SUMMARY

This disclosure pertains to a care plan delivery platform to enable wellness organizations to faithfully transmit their instructions to their patients and monitor progress. The care plan delivery platform includes: a backend engine to archive plural clinical protocols for a patient; an engine to escalate to help patients remediate non-compliance and address deviations from normal clinical parameters; a patient interface to prevent adverse situations; and interfaces for care providers, insurance companies, hospitals, patients, and other authorized users to interact with the platform.

The care plan delivery platform engine uses a uniform protocol definition framework to enable centralized development, transmission, modification, recording, and monitoring of multiple consolidated clinical protocols, and detection and controlled escalation to remediate non-compliance.

The platform comprises a protocol management system, whereby care providers may create and maintain databases of treatment protocols, such as general treatment protocols, protocol variations, and protocol implementations specific to individual patients or patient groups. All protocols may be stored in a standard clinical protocol format, which may include various elements, such as parameters to be tracked, actions to be completed, and situations and symptoms to watch for. Each protocol element may have a related schedule, normal and abnormal thresholds, procedural details, escalation instructions to remediate non-compliance as well as deviations from normal ranges, and associated instructions, training, and reference materials.

The protocol management system may be accessed by one or more portals, whereby authorized users, including care providers, may: create or maintain standard protocols, protocol elements, protocol variations, and protocol implementations specific to individual patients or patient groups; review, enter, or query observations and compliance data; monitor automated escalations and notifications; or institute a manual intervention or escalation. Entire protocols as well as protocol elements may be exchanged in a standard protocol format.

The protocol management system may be accessed by one or more patient portals, whereby patients or their proxies (such as home care providers, nurses, friends, and relations) may: receive care plan reminders; enter or modify data and observations, including responses to survey questions; certify compliance with protocol element requirements; receive training; provide consent (e.g., to participate in a research study); create new or modify existing protocol elements; and access reference materials. Such patient portals may be implemented, for example, as a graphical user interface, such as a web site, mobile computing application, or personal computer software.

The protocol management system may be accessed by one or more patient-side machine-to-machine portals, whereby machines affiliated with a patient may: receive care plan information; report interactions with the patients and their proxies; and report observations, such as clinical instrumentation readings. Such a machine-to-machine portal may operate to connect a personal computing device associated with a patient with the protocol management system, with or without the knowledge of the patient. For example, a glucose meter wirelessly connected to a patient's cell phone may report one or more glucose level observations to the platform, either as measurements are taken, periodically, or on demand from the protocol management system. Similarly, reminders may be delivered to the patients, or elements of protocols initiated automatically, by devices connected to the protocol management system via a machine-to-machine portal. The standard clinical protocol format may facilitate automation of the machine-to-machine exchange of protocol element information.

The protocol management system as well as the observations/data generated from its implementation may be accessed by an escalation control system, whereby the escalation system may receive information such as observations and data regarding a patient's health and compliance behaviors. By accessing the protocol management system, the escalation system may also: assess the patient information in the context of a consolidated care plan (consisting of multiple clinical protocols) being administered to the patient; recommend an escalation action designed to stimulate improved compliance with one or more protocols or protocol elements; and communicate with the patient or their proxy. Similarly, the escalation system may recommend or initiate a remedial action, such as an added protocol element, such as a medication substitution. Hence, the escalation system may act as an influence engine to help ensure adherence to the protocols that are tracked and monitored by the system.

The protocol management system and the escalation control system may be accessed by an artificial intelligence (AI) for purposes of: analyzing individual and population behaviors and outcomes; assessing efficacy of individual and multiple protocols, protocol elements, and escalation controls; automatically generating new protocols and proposing protocols or protocol elements for individual patients or patient groups; and/or tailoring new escalation controls based on outcomes of prior escalation controls. The AI may be trained with data, inter alia, from the platform, including the protocol management system and the escalation control system, as well as patient demographics and outcomes, general healthcare statistics, and protocol-specific healthcare statistics.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to limitations that solve any or all disadvantages noted in any part of this disclosure.

### BRIEF DESCRIPTION OF THE FIGURES

A more detailed understanding may be had from the following description, given by way of example in conjunction with the accompanying drawings.

FIG. 1 is a block diagram of an example compliance engine comprising a protocol management system, an escalation control system, portals, and other entities.

FIG. 2 is a call flow of an example technique of maintaining and monitoring progress in the implementation of various clinical treatment protocols and escalation protocols.

FIG. 3 is a call flow of an example technique of using an artificial intelligence in response to a set of conditions observed regarding a patient.

FIG. 4 is a representative embodiment of the patient mobile application showing the use of “cards” to remind patients to take actions and record parameters and the conversational AI avatar that engages with patients.

FIGS. 5A-D show examples of the many types of customizable cards that help patients capture data, complete activities, and learn information.

### DETAILED DESCRIPTION

### Challenges for Clinical Protocol Compliance

Certain problems are inherent in conventional approaches to clinical protocol management. Normally, diagnostic and treatment protocols are disseminated via scientific or medical journals, and then variably adopted and adapted by physicians for individual patients. Each of these protocols can range from dozens of pages to well over a hundred pages. No standard format has been agreed upon for publishing clinical protocols; each protocol has its own format and its own approach to sharing the best practice diagnostic and treatment information. No standard mechanism has been invented to record best practices codified by clinical protocols in a way that allows a machine to transmit instructions to patients and subsequently track whether the patient has complied with these instructions. Given this, there is also no way to track what instructions were given by care providers to their patients or how care providers adapted best practice guidelines for their patients. Moreover, since these guidelines are written for the population at large, the clinician is left to repeatedly customize the default protocol for each individual patient during each patient encounter. This introduces substantial variability and inconsistency in the care of patients and represents a substantial burden for care providers, who are “reinventing the wheel” during each patient encounter.

Furthermore, today, the patient is relied on heavily to comply with the care plan she received from her physician. This can be very difficult, especially as the patient deals with the stress of illness, disability, and the complexity of clinical regimens, in addition to the often-considerable challenges of daily living. Research confirms this fact, noting that the majority of patients forget up to 80% of physician instructions within an hour of the interaction. Worsening this issue, limited and expensive healthcare resources cannot be made available all day to support and answer patient questions, largely leaving them and their families to rely on themselves to manage their care.

These factors have contributed to worsening patient compliance to care plans—the instructions their physicians would like for them to follow to get healthy and stay that way. In summary, many factors play a role in driving patient non-compliance to care plans: growing complexity of care plans, due in part to increasing number of conditions that patients are diagnosed with; inability to reconcile the instructions from multiple different physicians; patient forgetfulness; care plan side effects; and high costs of compliance (e.g., medications).

The impact of non-compliance to care plans is growing. Though non-compliance is not the only factor driving increased healthcare spending, it represents a meaningful contributor to the increase in US healthcare spending from $3T in 2017 to a projected $4T in 2021.

### Solution Advantages

The compliance engines described herein address many of the shortcomings of traditional management of multi-comorbid patients, who are charged with following multiple clinical protocols. The protocol management system affords a consolidated library of protocols for any condition. The care provider portal enables the user to manage a multi-comorbid patient by selecting all the relevant protocols, customizing the protocol elements, and deploying the resulting care plan to the patient. This allows the provider to faithfully transmit his or her instructions directly to the patient, ensuring the patient knows exactly what he or she must do. The patient portal receives these instructions and provides the patient with a unified view of the provider's instructions, alleviating many of the challenges that drive non-compliance. The machine-to-machine portal allows easy integration of tools for communication with patients and monitoring equipment. Working with all of these components, the escalation control system provides important automated oversight and support.

In short, the compliance engine provides a way to pierce information barriers that arise from the natural complexity of multi-faceted clinical care, and provide patients and care providers with a simpler way to align on what must be done to ensure effective health maintenance and respond to contingencies. Together, these should drive better healthcare outcomes, and over time reduce overall healthcare spending.

### General Remarks

Herein, the term “technique” refers to ways of using information technologies to achieve particular ends. The term “technique” is generally used in place of “procedure” and “operation” to avoid confusion with clinical procedures, and in place of “method” to avoid confusion with special meanings of the term “method” in the context of machine-to-machine and Internet-of-things applications. The steps described for techniques herein are often optional, and may potentially be performed in a variety of ways and a variety of sequences. Hence, herein the term “technique” should not be interpreted as referring to a rigid set and sequence of steps, but rather to a general methodology for achieving results that may be adapted in a variety of ways.

Herein, the terms “care plan,” “protocol,” and “clinical protocol” may be used interchangeably and refer generally to overall plans, or portions of overall plans, designed to assist the wellness of individuals, and may include medical, clinical, fitness, social activities, and emotional and psychological therapies, for instance, as may be created to support the overall wellness of a patient or other participant in a monitored program. Hence, for example, the system may store and act on a “protocol” designed to support an elderly patient with a memory disorder which includes such disparate elements as daily medication dosing, home blood pressure monitoring, yoga, daily walks, regular doctor appointments, support group attendance, and home care (e.g., securing one's home before bed). Associated escalation mechanisms supported by the system may include, for example, reaching out to caregivers, family members, social workers, counselors, etc., as may be required, e.g., due to the diminished capacities of the individual being treated. Orchestrating, monitoring, and trouble-shooting the wellness of an individual are complex challenges, and a wide variety of facets of the solution; from the medical to the social; are addressed by the protocol systems, methods, and apparatuses described herein.

Note that protocols may be designed and tailored at many levels of abstraction. At the highest level of abstraction, a protocol may capture, for example, a regional policy for the treatment of a condition or preventative protocol for an at-risk population. A separate version of the protocol may be tailored for an individual facility or medical practice. Similarly, versions may be created to address different demographics or combinations of conditions, or both. Ultimately, protocols may be tailored for individuals, and may vary over time for the same individual, e.g., with alternate protocols on file for times of chemotherapy during active treatment vs. recovery and remission.

### Example Architecture

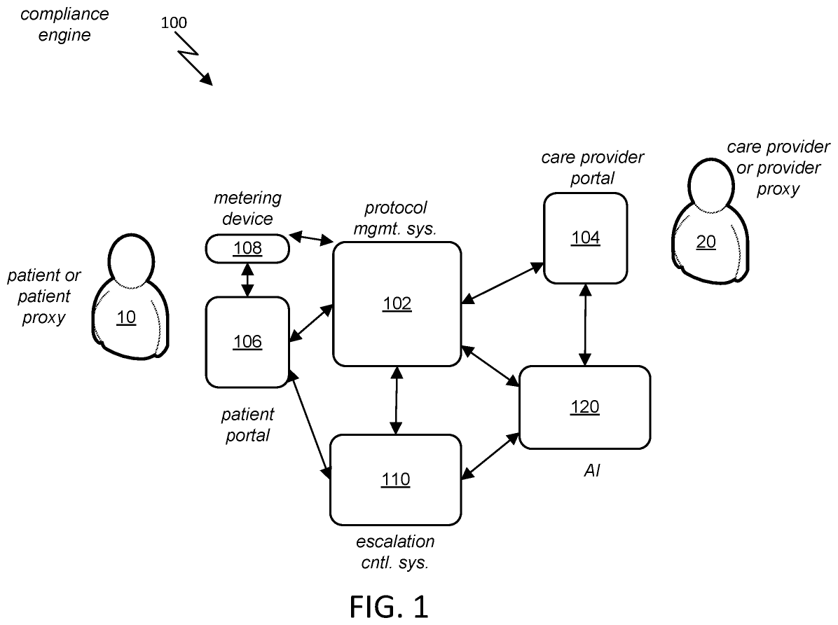

FIG. 1 is a block diagram of an example care plan delivery engine 100. The care plan delivery engine 100 comprises a protocol management system 102, care provider portal 104, patient portal 106, escalation control system 110, and an artificial intelligence (AI) 120. In practice such a system may contain any number of elements. Such a system may be used to interact with any number of patients 10 and care providers 20, and their proxies. Similarly, such a system may interact with any number of metering devices 108 that are associated with individual patients. For example, there may be many care providers 20 and many care provider portals 104 to interact with care providers and their proxies, such as doctors, hospital administrators, health plan executives, laboratories, therapists, school clinical staff, residential staff, nursing staff, and home care providers. Similarly, there may be many patient portals 106, e.g., for patient mobile devices, email, text messaging, and web interface for use by patients and their family, friends, and in-home caregivers. Similarly, there may be many metering devices 108 associated with each patient, including blood sugar glucometers, blood pressure meters, pulse oximeters, motion detectors, sleep monitors, spirometers, heart rate and heart rhythm monitors, and pedometers, as well as other clinical devices such as pumps, ports, and implants such as cochlear, nerve stimulus, and pace maker devices. Metering devices 108 may be assigned to a patient permanently or temporarily, e.g., at a gym or a clinical facility during a checkup, treatment session, or stay.

The systems, apparatuses, and methods described herein may be implemented in a wide variety of ways. For example, each of the functionalities represented in FIG. 1 could be divided or combined in a number of ways. For example, the protocol management system 102 could include separate databases for protocols as they are designed and approved, a second database recording interactions with patients, and a third recording noted deviations from protocols or issues in confirming compliance.

### Example Technique

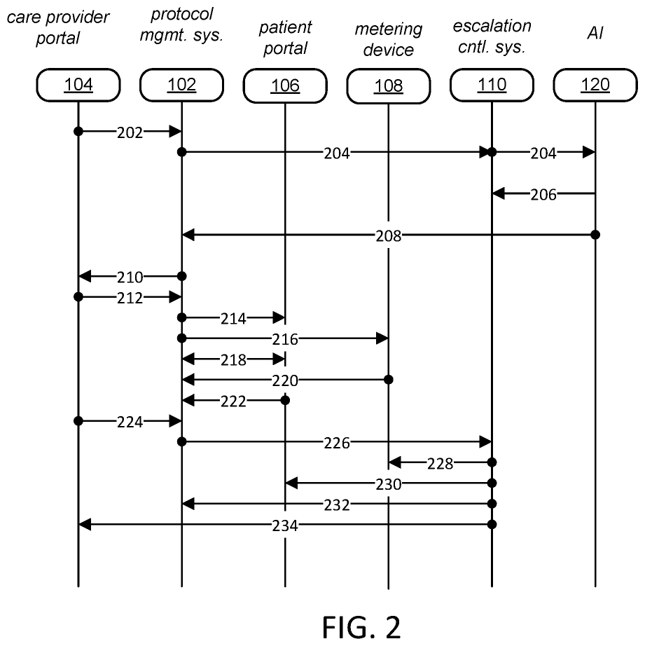

FIG. 2 is a flow diagram of example techniques for the use the care plan delivery engine 100 of FIG. 1. In step 202, the care provider portal 104 sends a general treatment protocol to the protocol management system 102. The protocol management system 102 stores the protocol in a standard clinical protocol format that is common across conditions. The general treatment protocol, in the standard clinical treatment protocol format, comprises a set of elements. These elements may include parameters to be tracked, actions to be taken, and/or symptoms or situations to watch for. The parameter to be tracked is further defined by the schedule the patient should follow to track this parameter, the normal and abnormal ranges for this parameter, as well as a set of escalation paths to follow when the measured parameter is out of the normal range and when it hasn't been measured according to the prescribed schedule. The actions to be taken are further defined by the schedule the patient should follow in performing these actions as well as a set of escalation paths to follow when the actions are not completed according to the prescribed schedule. The symptoms to watch out for are further defined by a lay person description of how the patient may feel if she were to be experiencing that situation so that she is able to self-evaluate her present condition, as well as a set of actions for the system to instruct the patient to take if she were experiencing that situation.

In step 204, the protocol management system 102 may forward the standard protocol information to the escalation control system 110 and the AI 120. For example, the escalation system 110 may store the information in order to compare future patient data to a planned regimen. The AI 120 may use general treatment protocol information to learn patterns of treating plans generally and as to specific clinical treatment indications. For example, the AI 120 may look at data for large populations while considering data about an individual to determine escalation for that individual. Conversely, the AI 120 may similarly consider changes to general protocols based on observation of compliance in more specific protocols or in the cases of individuals.

In step 206, based on the general treatment protocol and other information, the AI 120 may send a proposed escalation protocol for the general treatment protocol. The protocol may include non-clinical practices that do not require doctor or FDA approval, for example. However, an AI such as AI 120 may optionally be qualified by the FDA as a clinical device to provide a clinical recommendation. Herein, all AI recommendations are presumed to be either rendered by a qualified clinical device, subject to approval by one or more physicians, or non-clinical in nature.

In step 208, the AI 120 may make a recommendation for a new protocol or modification to an existing protocol. Again, as discussed in reference to step 206, such a recommendation herein is presumed to be either rendered by a qualified clinical device, subject to approval by one or more physicians, or non-clinical in nature. The recommendation in step 208 may be, for example, a recommendation for a combined protocol for the management of multiple conditions suffered in a patient.

In step 210, the protocol management system provides a copy of a general treatment protocol on request to the care provider portal 104.

In step 212, the care provider portal 104 provides a version of the general protocol that is tailored for use by a specific patient. The tailored protocol is stored in the standard clinical protocol format as a new version.

In step 214, the protocol management system makes this protocol available to the patient and others via the patient portal 106.

In step 216, the protocol management system also makes portions of the tailored protocol available to metering devices associated with the patient. For example, such portions of the protocol may include medication dose amounts and schedules, contingent dosing information, schedules or frequencies for the collection and reporting of data, limits of expected readings, and contingent actions and reporting to be undertaken when readings are beyond normal limits. Notably, such information may be delivered automatically to the metering device 108 due to the maintenance of the tailored protocol in the standard clinical protocol format.

In step 218, the patient portal 106 and the protocol management system 102 may interact to execute and monitor patient training and motivation activities to initiate the tailored treatment protocol.

In step 220, the protocol management system 102 receives patient data from the metering device 108. The protocol management system 102 may then compare such data to the tailored, individual patient protocol to detect potential issues such as non-compliance, a value outside of the normal range for that patient, or a change in the patient's condition.

Similarly, in step 222 the protocol management system 102 receives patient data from the patient portal 106, which may include, for example, patient inputs such as a symptom or a failure to make appointment.

Further, in step 224 the protocol management system 102 receives data from the care provider portal 104. Such data may include, for example, a failure to make appointment, a diagnostic observation, or a laboratory result.

In step 226, the protocol management system 102 may share some or all of the data associated with the patient that is learned in steps 218, 220, 222, and 224.

In steps 228, 230, 232, and 234, the escalation control system 110 may send a response to one or more of the metering device 108, patient portal 106, protocol management system 102, and care provider portal 104. The escalation control system 110 may take a number of actions based on the information received in step 226, the tailored protocol for the associated patient, or any subsidiary escalation protocol associated with the patient treatment protocol. For example, the escalation control system 110 may recommend a different metering schedule to the metering device 108, send a message to the patient via the patient portal 106, trigger the logging of an event or protocol change by the protocol management system 102, or set a target new appointment date to the care provider portal.

### Example Technique Using AI

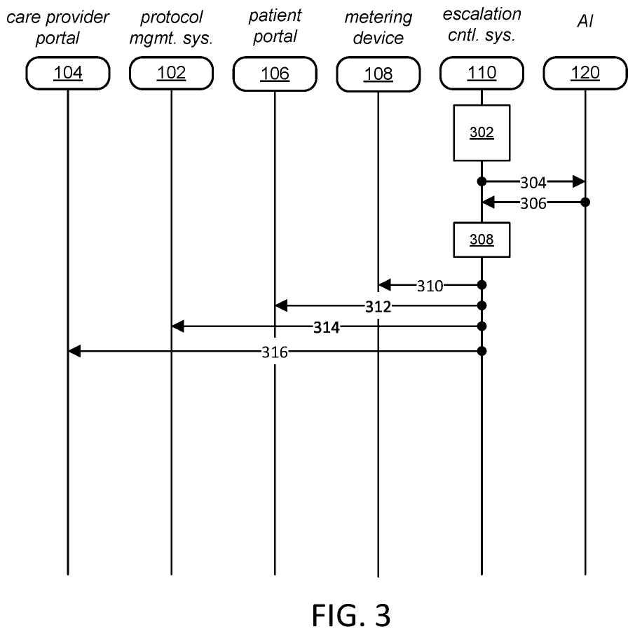

FIG. 3 is a flow diagram of an alternative response by the escalation control system 110 to the information received in step 226. In step 302, the escalation control system 110 considers the information received in step 226 in view of a broader context of, for example, the history with the particular patient, patient demographic, and/or clinical indication or combination of indications.

If the escalation control system 110 considers the normal treatment or escalation protocol responses to be ineffective, e.g., via repeated non-compliance, the escalation control system 110 may consult the AI 120 in step 304. In practice, an assessment of the efficacy of outbound interactions may be made by the AI 120 on an ongoing basis, with or without input from the escalation control system 110.

In step 306, the AI 120 may respond with a new or altered protocol intended to motivate more desirable results in the patient. This may arise, for example, from indirect observations of the patient or the patient's cohort or demographic, e.g., via population clinical information, social media observations, and/or other sources.

In step 308, the escalation control system 110 processes the recommendation from the AI 120 in step 306. This may entail altering a clinical treatment protocol or non-clinical protocol. Then in steps 310, 312, 314, and 316, the escalation control system may propagate recommendations and/or revised protocols to one or more of the metering devices 108, patient portal 106, protocol management system 102, and care provider portal 104.

It will be appreciated that the system functionalities described herein may be physically arranged in any number of ways. For example, the protocol management system and escalation control system may be two aspects of the functionality of a single server or server cluster, or replicated on many servers and server clusters. Similarly, a patient portal may manage communications with metering devices as well as with patients and patient proxies. Any system element can be tasked with detecting non-compliance, changes in patient condition, or other events. The examples of FIGS. 1, 2, and 3 will be understood to be just a few of many equivalent configurations available for implementation.

### Example Variations

The use of a standard clinical protocol format enables a universal framework for organization of care plans across clinical conditions. A standard clinical protocol format may include, for each protocol, a set of elements made up of parameters to be tracked, actions to be taken, symptoms to watch for, and a period of time for each parameter to be tracked, action to be taken, and symptom to watch for. In other words, all kinds of treatment—from care of chronic diseases (e.g., diabetes, hypertension, and rheumatoid arthritis) to acute procedures (e.g., orthopedic, dermatologic, and surgical) and anything in between (e.g., cancer care) may be accommodated in a single system. Further, by standardizing regimen descriptions, e.g., the details and timing of actions to be taken and parameter measurements, for example, the use of a standard clinical protocol format facilitates automation of protocol dissemination and compliance monitoring. The standard format makes the protocols machine-actionable. These facets may similarly be joined with automation of maintenance and remedial action escalation, such as patient communications and motivation.

For example, based on instructions defined by the provider, a protocol management system may create a rules-based, machine-actionable, personalized health plan for each patient. An escalation system may then influence the patient or caregiver to complete the items in the personalized health plan. Escalations may involve outbound interactions with the patient, e.g., when she fails to mark care plan items as complete, or when excursions beyond normal thresholds occur for measured parameters.

The escalation control system may engage the patient directly through a multi-channel, operating-system agnostic approach, such as via SMS messaging, in-app calls, voice or video calls to the mobile phone, voice calls to the home phone line, and messaging smart devices such as smart speakers or smart TVs. If the escalation control system is unable to reach the patient directly, the system may be programmed to engage friends and family, e.g., as indicated by the patient during her on-boarding, and her care providers. Each potential outbound interaction may follow one of several different escalation paths, with each progressively and appropriately aligned to the importance of the message (as judged by the patient's care team) to increase the “nudges” used to improve compliance.

Because patients and their friends and family will not want unlimited outbound interactions from the system, the escalation control system may use a general limit to the number of outbound interactions that the system initiates to any individual on any given day. Further, to ensure the patient follows the care plan to maintain/improve health and avoid complications/emergencies, outbound interactions may be classified in various categories, such as routine and exceptional, based on the urgency/impact of the outbound interaction on the patient's health. For example, routine items may be constrained by the general daily outbound interaction limit, while exceptional items may be able to exceed this limit, enabling the system to reach the patient for important, urgent, high impact items. Default designations may be offered in the standard protocols, but these can be modified by the patient's care team, according to the clinical judgment.

### Parameters

Normally, each protocol in standard clinical protocol format will include one or more parameters to be measured. These may include any of a wide range of quantitative and qualitative dimensions that can be used to assess a patient's health. A care plan may include parameters to measure, measurement frequency, when and how to take measurements, and “normal” boundaries within which each of these parameters should fall.

Measured parameters may include biometrics that a patient or a patient's metering device may be able to determine in the patient's home (e.g., heart rate, blood sugar, and blood pressure), and biometrics that may be derived in a laboratory from a sample taken from a patient (e.g., blood electrolytes or urine albumin.) Measured parameters may include subjective scores that a patient or caregiver is asked to report, such as pain level and appetite. Measured parameters may also include, for example, diagnostic information derived using at home or in-facility based clinical equipment (e.g., heart rhythm, electrocardiogram, and chest x-ray results.)

Measured parameters may have ranges for what is considered to be normal, low, or high. For example, the normal range for blood glucose may be designated as anything reading with values between 80 and 130. Values lower than 80 are considered low and remedial actions should be taken to bring the patient's blood glucose back to normal range. Values higher than 130 are considered high and other remedial actions should be taken. In one embodiment of this invention, each parameter have may a designated normal range, three low ranges (low 1, low 2, and low 3) corresponding to increasing levels of clinical severity associated with lower than normal reading and the corresponding remedial action required, and three high ranges (high 1, high 2, and high 3) corresponding to increasing severity associated with higher than normal readings and the required remedial actions.

### Actions to Take

Normally, each protocol in the standard clinical protocol format will include one or more actions to be taken. These may include actions that the patient must complete herself, such as changing a dressing or exercising for 30 minutes. The actions may also include things that a patient may only complete with the help of another individual or a professional, such as receiving nutritional counseling or conducting a neurologic examination

### Symptoms to Watch for

Normally, each protocol in the standard clinical protocol format will include one or more symptoms to watch for. A healthcare provider should advise the patient about potentially dangerous situations that may arise due to her illness. These situations are typically accompanied or preceded by some markers about which the patient should be vigilant.

For example, a patient may be asked to watch out for heart attack. This watch may be assigned to a patient with coronary artery disease. Another patient may be told to watch out for deep vein thrombosis, which has increased prevalence in those who have had surgery or suffer cancer.

### Tailoring Protocols

A general clinical treatment protocol may be tailored for individual patients at the discretion, for example, of a treating physician, or in view of the success or failure of efforts to remediate non-compliance or shifting patient condition. Tailoring an individual's protocol may involve, for example, adding or removing elements of the care plan, such as whether the patient needs to measure glucose measurements. Similarly, tailoring may involve changing the frequency with which he asks the patient to do any element of the care plan, or may involve modifying the thresholds beyond which the patient must take action. For example, a systolic blood pressure of 140 mm Hg may be high for one patient, while 130 mm Hg may be the limit for another.

### Automated Oversight

Digitization of protocols into a standard clinical protocol format is essential for automated oversight. By placing a care plan into the standard clinical protocol format, digital agents, e.g., processes or “bots” active in the protocol management system, escalation control system, patient portal, care provider portal, or even metering devices, may periodically or continuously compare each patient's care plan with the available data feed from other system elements regarding measurements, actions, and observations. Digital agents may remind patients and care providers to complete measurements and take actions at the right times. As measured values are reported, the system compares the resulting measure with the limits defined by the healthcare provider for this patient. When there is a deviation of the value from the limits defined by the healthcare provider, agents may trigger escalation responses.

### Digitization and Multi-Indication Complexity

The use of a standard clinical protocol format has surprising effects on the treatment of patients with multiple indications. This is because centralization and harmonization of the care plan, along with integration with patient portals and metering devices, helps to overcome traditional difficulties faced in particular by patient who suffer from multiple clinical conditions.

First, patients often have difficulty understanding all the different parts of a complex care plan. Even the most basic care plan requires a lot of the patient. The complexity increases even more when that patient has been diagnosed with several illnesses.

Second, patients have difficulty remembering to do the things on the care plan at the prescribed time, if at all. Patients find it difficult to remember to integrate the care plan elements into their already busy lives

Third, patients suffer from a lack of a readily accessible reference for the plan. Traditionally, a patient may have a variety of healthcare providers, each of whom tailors a different care plan for the patient, and these are provided manually. As a result, the patient has no single, central place to turn to remind her what she must do. Similarly, the care providers may have no coherent, central repository of information regarding what each specialist may be advising the patient.

Therefore, the use of a central protocol management system that unites plural care providers and protocols in a central standard format not only enables a variety of digital tools for facilitating compliance, but it also solves human factor difficulties in streamlining communications and providing confidence in the sources of information.

### Considerations for the Escalation Control System

Three situations that an escalation control system would typically address are a missed parameter measurement, a missed or incomplete action, and a deviation of a parameter outside of the normal range. In response, the escalation control system may provide a simple communication, a “nudge”, to remind a patient, for example, to take a measurement, take a medication, take an action, or finish an action started but not completed.

However, nudging is only effective when the effort expended to reach the patient or care provider is proportional with the urgency of the situation. For example, it is counterproductive to call a well-controlled diabetic repeatedly to remind her to measure her blood glucose level. Good matching is needed to ensure that the patient does not “tune out” the information received from the system.

Similarly, nudging is not productive when the patient is already engaged on the platform, such as when they are reporting a symptom. In such a case, we already have her attention.

In addition to the current level of engagement of the patient with the system, the seriousness of the situation may drive who is involved in the escalation path, how quickly, and with what effort, persistence, and speed. Often, it is the patient herself who needs to be informed. However, the patient's proxies and caregivers may also need to be informed, depending on circumstances. The most likely impact of such an event, whether it is non-compliance with a care plan item or a deviation of a value outside the normal range, on the patient's health (e.g., how long before the patient's health is impacted by this event) may determine which escalation path is most appropriate, and how quickly such a path should be pursued.

The escalation control system may accommodate situations in which a simple nudge is insufficient to get the patient to address the issue, whether it is remediating non-compliance to a care plan element or taking action on the deviation of a measurement outside the normal range. Multiple escalation actions may need to be taken depending on the likely severity of the impact to the patient's health as well as the speed to the impact. For each escalation action, a different stakeholder may be the intended point of contact, and different time intervals may be specified.

### Example Data

Table 1 of the Appendix shows an example tailored clinical treatment protocol alongside associated compliance data and data linkages. In the example of Table 1, a patient with Type II diabetes and arthritis is prescribed seven protocol elements that include metering, actions to be taken, and symptoms to watch for. Each element is shown as a separate item in rows 1-7. Each element of the protocol has associated type, sub-type, detail, frequency, and schedule information in columns A-E.

Columns F and G of Table 1 contain current compliance information related to each element of the protocol, and columns H and I contain data link information associated with the protocol. Table 1 represents an example intelligent patient chart which, in addition to capturing current treatment plans and statuses for the patient, illustrates how such information may be standardized to make the information machine-actionable, and to be more readily accessible for human users.

For example, Item 1 of the protocol is shown in columns A-E as the in-home metering of blood glucose levels, which is intended to occur twice daily, prior to taking medications at morning and evening meals. Columns F and G show that the latest reading required, the morning reading, was taken properly, but the measured result is outside of the recommended limits. For simplicity, such details as the taking of meds at meals and the exact range expected are not shown in Table 1. However, it will be appreciated that such information may be included in the protocol to inform the patient and to allow automated evaluation of metering results, for example.

Columns H and I of row 1 enable the compliance engine to obtain the in-home metering information directly. For example, the meter used may be a smart device with direct or indirect links to the Internet, e.g., directly via a cellular connection, or indirectly via the patient's cell phone over a WiFi or Bluetooth connection. By standardizing the format of resultant data, range limits, and rules, the meter data becomes machine-actionable. Similarly, by pairing device information with protocol elements, machine-to-machine communications can resolve many issues that previously would require human intervention.

In the example of Table 1, the data structures are shown in tabular form. In practice, any data structure may be used. For example, relational databases may be used, allowing great flexibility in what kind of information is stored for various protocol elements, compliance information, and links associated with compliance and protocol elements.

It will be appreciated that the various data components may be distributed in various locations in a care plan delivery engine. For example, the protocol, compliance, and data link information may all be maintained by a protocol management system. Alternatively, the protocol management system may be tasked only with maintaining the official protocol, while an escalation control system monitors and records compliance data, and a patient portal system attends to details of data links.

Row 2 provides an example of another kind of metering which, currently, must be performed by a laboratory on a blood sample provided by the patient. In this example, the testing has been completed, but again the result falls outside the guideline set by the protocol. The data link information here is shown as the lab providing the results and the associated lab order number. Additionally or alternatively, not shown, the data link may provide reference and training links, e.g., for use by the patient to better understand the testing, or by a clinician to drill into details of test methodology used by a particular lab.

Row 3 illustrates an example of a non-clinical aspect of the protocol useful in educating and motivating the patient. Like the other elements of the protocol, the initial diet counseling of this example may be tracked by machine interfaces to automate compliance checking and remediation. Links to the relevant information are part of the overall record, and therefore remain available to the patient centrally throughout the course of treatment.

Row 4 illustrates perhaps the most familiar aspect of a tailored clinical treatment protocol, which is prescribed medication. Beyond tracking what the prescription is, the compliance engine of the example of Table 1 is also tracking compliance in taking the medication and providing persistent links to be available to the patient in case they have questions. Similarly, the compliance engine could be configured to monitor other aspects of the compliance of this element of the protocol, such as whether prescriptions have been filled and retrieved promptly by the patient from the pharmacy.

Row 5 shows a protocol element of exercise. In the example shown, the data links are related to educational suggested guidelines. Additionally or alternatively, not shown, the data links could include a connection to a patient device, such as a pedometer or exercise monitoring function on the patient's smart phone, allowing data to be collected without specific separate data entry by the patient.

Row 6 shows an example symptom for which the patient should be on guard. In this example, the symptom is the dizziness that may accompany a low blood sugar level, e.g., in the case of overmedication via insulin.

Row 7 shows the integration in the protocol of elements intended to address a different clinical condition. In this example, the patient is merely asked to report once daily at a consistent time about the level of discomfort associated with the illness. Links to training and/or reference materials about that illness are also provided.

Table 2 shows an example of data that may be associated with the patient illustrated in the example of Table 1. Here in Table 2, the escalation status and history are maintained. This includes the situation, history of included and outbound contacts, a remediation recommendation, a determination of the present urgency of the situation, and the next actions to be taken.

In addition to the information not shown in Table 2, the system may include rules and mechanisms to reach such conclusions, e.g., based on the protocol and compliance status data of Table 1. In some cases, such determinations may be simple and machine-actionable by ordinary computing means. In other cases, the determinations may be complex and require human judgement, the invocation of an artificial intelligence, or both.

In the example of Table 1, as reflected in the determinations of Table 2, the patient's blood glucose has apparently remained out of range, in both the last laboratory report and today's morning reading. If both readings were out of range on the high side, it may be presumed that the medication level is too low, or that the patient's diet and activity levels are not under proper control. This may suggest that a checkup visit to a clinician would be helpful. If the morning reading were too low, in addition to recommending a checkup, the system may also wish to prompt the patient regarding dizziness, which so far, the patient has not reported.

Additional reminders to the patient may not be in order. The patient was reminded to take meds, and did, this morning. The patient was also in contact with the system to report that morning exercise was being skipped. Therefore, this is not a case of the patient being non-responsive to system prompts. Therefore, unless the morning meter reading is dangerously high, the system will not recommend urgently contacting the patient, physician, or a patient's proxy. Therefore, the urgency or escalation level in Table 2 is set to routine.

However, the system has detected a pattern for which some remedial action—scheduling a clinician appointment, is warranted. Notably this is a machine-actionable event, since the nature of the circumstances and the parties to contact are known. For example, the system may have access to the calendars of the responsible clinician, the patient, or both, and can broker an appointment time rather than, for example, simply urging the patient to call the clinician and wait on hold in an attempt to schedule an appointment time. Further, by participating in creating the appointment, the system can be informed that the required remediation has been scheduled, and consider this information when processing further status data regarding the client.

Table 3 in the Appendix shows an example of data that may be associated with the escalation paths used by the escalation control system. In the example of Table 3, example escalation paths are defined in terms of whom to contact first, whom to contact next, and when to begin the next contact if the first contact does not resolve the issue. Escalation paths may be defined in a number of ways, including, but not limited to, the severity of issues and the timing of attempted resolution steps. Similarly, each step may be triggered on various criteria, such as timing and severity, and combinations thereof.

Table 4 of the Appendix shows an example protocols for treating various conditions. In the example of Table 4, a number of parameters, actions, and “watch outs”, things to watch out for, are associated with each protocol. For example, the protocol CSVR CAD v1, which may be used for patients with coronary artery disease, has two parameters to be tracked, six scheduled actions to be taken, and three issues to watch out for. Each of the parameters, actions, and watch outs for each protocol in Table 4 may have an associated set of protocol, compliance, and link details, e.g., such as is illustrated or protocol elements in Table 1. Each parameter, action, and watch out may have any associated range of thresholds, as illustrated in Table 5, for determining the potential impact of each measured parameter, action, or symptom, or example. Data on parameters, actions, and watch outs, may trigger various escalation steps, as shown, for example, in Table 3. The efficacy of escalation actions may be tracked, as illustrated in the example of Table 2.

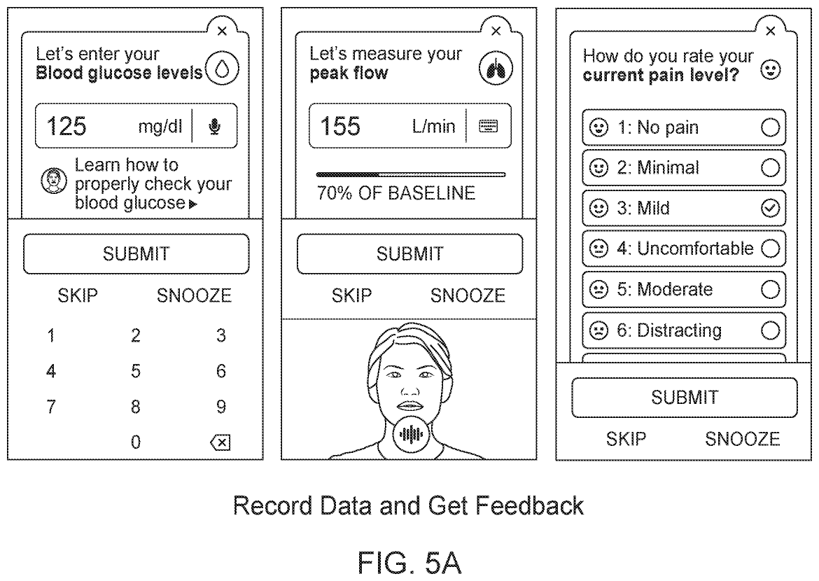

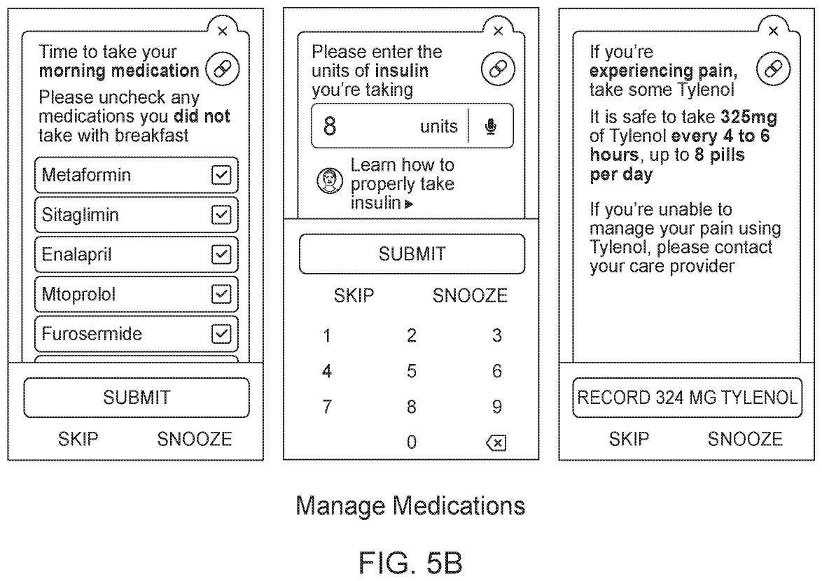

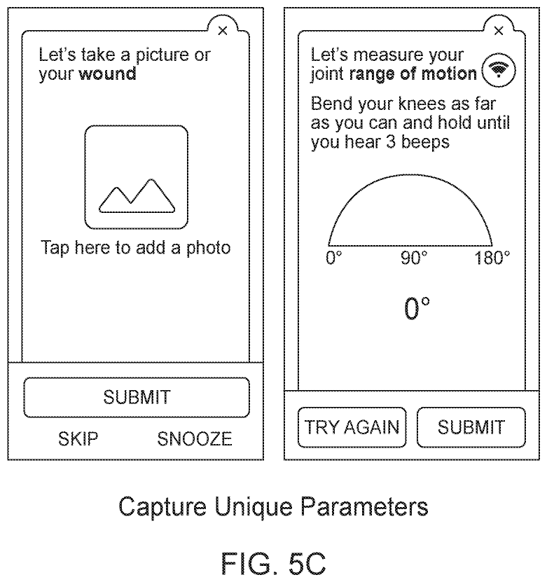

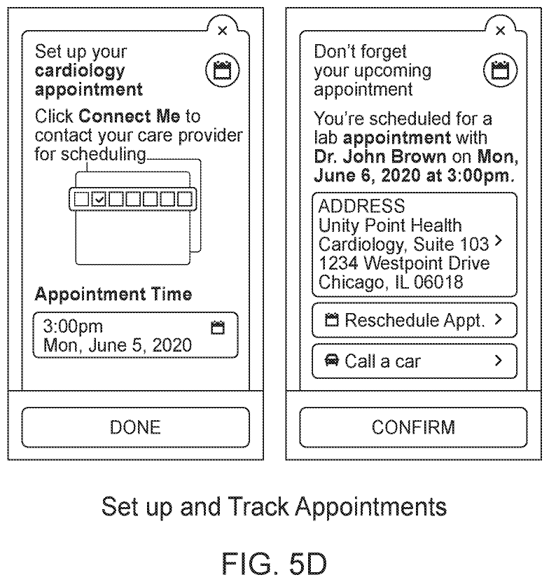

Table 5 of the Appendix shows a variety of different escalation actions that may be taken in response to reported conditions, or the failure to report conditions. In the example of FIG. 5, the selection of escalation steps is based on a parameter monitored for the patient, and escalations to the same parties may be classified as exception or routine, for example, depending on the urgency of the situation.

### Example Interactions

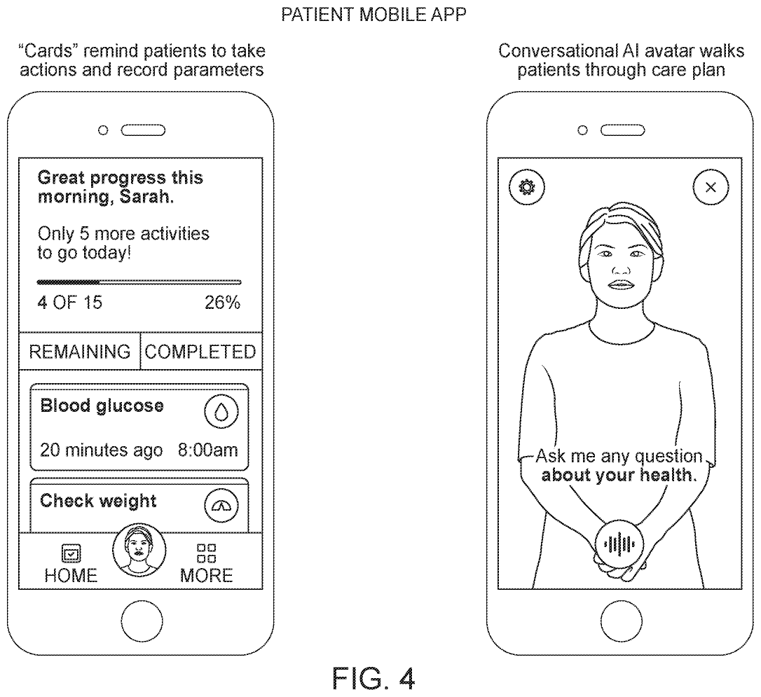

FIG. 4 illustrates an example of interactions a patient may have with a care plan system via a mobile device such as a cell phone. On the left, a patient mobile application shows a “card”, a digital reminder, to alert the patients to take an action or record a parameter. On the right, the user's experience is an enhanced via interaction with a conversational AI avatar that engages with the patient.

FIGS. 5A-D show examples of the many types of customizable cards that may help patients capture data, complete activities, and learn information, e.g., via a patient mobile device application. FIG. 5A illustrates patient mobile device interfaces for recording data and getting feedback. FIG. 5B illustrates interfaces for medication management, and FIG. 5C illustrates interfaces for capturing unique parameters, such as wound images and range of motion data. FIG. 5D illustrates interfaces for making, managing, and tracking appointments.

### Technical Implementation

It is understood that any or all of the techniques, systems, methods, devices, and processes described herein may be embodied in the form of computer executable instructions (e.g., program code) stored on a computer-readable storage medium which instructions, when executed by a machine such as an apparatus of a mobile computing device, computer, server, gateway, or other computing device, perform and/or implement the systems, methods, and processes described herein. Specifically, any of the steps, techniques, or functions described above may be implemented in the form of such computer executable instructions. Computer readable storage media may include both volatile and nonvolatile, removable, and non-removable media implemented in any non-transitory (i.e., tangible or physical) method or technology for storage of information. Such computer readable storage media do not include signals. Computer readable storage media include, but are not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other tangible or physical medium which may be used to store the desired information and which may be accessed by a computer.

TABLE 1
Tailored Protocol, Compliance, and Information Linkages

| | Protocol | | | | | Compliance | | Links | |
|---|---|---|---|---|---|---|---|---|---|
| | A | B | C | D | E | F | G | H | I |
| Item | Type | Sub | Detail | Frequency | Schedule | Status | Result | Data Link | Detail |
| 1 | meter | in-home | blood | 2 × daily | AM, PM; | AM | out of | meter s/n 1234; | URL: // |
| | | | glucose | | pre-med | complete | range | brand X | xyz.efg.com |
| 2 | meter | lab | A1C | every 3 | Feb. 1, | complete | out of | Lab Co., Inc. | order |
| | | | | mos. | 2019 | | range | | 789123 |
| 3 | action | training | diet | program | May 1, | complete | — | Mercy Hosp. | Course 1726 |
| | | | counseling | onset | 2017 | | | online | |
| 4 | action | med | metformin, | 2 × daily | AM, PM; | AM | compliant | ref. materials at | metformin |
| | | | 500 mg | | post-meter | complete | | PharmaXYZ.com | FAQ |
| 5 | action | exercise | 30 min lt. | 2 × daily | AM, PM | AM skipped | non- | Mercy Hosp. | exercise |
| | | | cardio, | | | | compliant | online | required. |
| 6 | symptom | diabetes | dizziness | as req. | as req. | none | — | Clinic Y | lo/hi |
| | | | | | | reported | | guidelines | glucose |
| 7 | symptom | arthritis | pain level | 1 × daily | 10 AM | complete | 4 out of | Clinic Y | arthritis |
| | | | | | | | 10 | guidelines | management |

TABLE 2
Escalation Status and History

| | |
|---|---|
| Situation | glucose remains out of range with |
| | compliant med use; exercise skipped |
| Last Outbound Contact | AM med reminder |
| Last Inbound Contact | AM exercise skipped |
| Remediation Recommendation | schedule checkup/adjust meds? |
| Urgency/Escalation | routine |
| Next Actions | determine physician availability |
| | match to patient availability |
| | make appointment |

TABLE 3
Escalation paths

| | | First Person | Next to reach | | |
|---|---|---|---|---|---|
| # | Escalation name | to Reach | Limit | Period | Whom to reach |
| 1 | No escalation | — | — | — | — |
| 2 | Remind Patient Once | Patient | 15 | Minute(s) | Patient |
| 3 | Notify Care Circle Member 1, then 2 | Patient | 15 | Minute(s) | Patient |
| | | | 1 | Hour(s) | Care circle 1 |
| | | | 1 | Hour(s) | Care circle 1 |
| | | | 2 | Hour(s) | Care circle 2 |
| | | | 2 | Hour(s) | Care circle 2 |
| 4 | Notify Practice via Portal | Patient | 12 | Hour(s) | Physician office |
| 5 | Notify Care Circle Member 1 | Patient | 1 | Hour(s) | Patient |
| | | | 1 | Hour(s) | Care circle 1 |
| | | | 2 | Hour(s) | Care circle 1 |
| 6 | Notify Care Circle Member 1, then | Patient | 6 | Hour(s) | Patient |
| | Practice/Call Center | | 12 | Hour(s) | Care circle 1 |
| | | | 24 | Hour(s) | Call center |
| | | | 1 | Minute(s) | Physician office |
| 7 | Quickly Escalate to Care Circle, then Call | Patient | 5 | Minute(s) | Patient |
| | Center, then EMS | | 5 | Minute(s) | Care circle 1 |
| | | | 5 | Minute(s) | Care circle 1 |
| | | | 10 | Minute(s) | Call center |
| | | | 1 | Minute(s) | Physician office |
| | | | 9 | Minute(s) | Emergency services |
| 8 | Notify Care Circle Member 1, then 2, then | Patient | 15 | Minute(s) | Patient |
| | Practice | | 1 | Hour(s) | Care circle 1 |
| | | | 1 | Hour(s) | Care circle 1 |
| | | | 2 | Hour(s) | Care circle 2 |
| | | | 2 | Hour(s) | Care circle 2 |
| | | | 1 | Minute(s) | Physician office |
| 9 | Notify Care Circle Member Immediately | Patient | 1 | Minute(s) | Care circle 1 |
| | | | 1 | Minute(s) | Care circle 2 |
| | | | 5 | Minute(s) | Call center |

TABLE 4
Managed Protocols

| | | No. of | No. of | No. of |
|---|---|---|---|---|
| Protocol Name | Condition | Parameters | Actions | Watch outs |
| CVSR Diabetes v1 | Diabetes | 3 | 5 | 3 |
| CSVR CAD v1 | Coronary Artery Disease | 2 | 6 | 3 |
| CSVR Hypercholesterolemia v1 | Hypercholesterolemia | 0 | 7 | 0 |
| CSVR Knee Replace v1 | Knee Replacement | 3 | 17 | 4 |
| CSVR Pregnancy v1 | Pregnancy | 1 | 8 | 4 |
| CSVR Hip Replace v1 | Hip Replacement | 2 | 17 | 4 |
| CSVR CABG v1 | Coronary Artery Bypass Graft | 5 | 21 | 7 |
| CSVR Asthma v1 | Asthmas | 3 | 6 | 1 |
| CSVR HTN v1 | Hypertension | 1 | 5 | 6 |
| CSVR Heart Failure | Heart Failure | 7 | 9 | 5 |
| CVSR PCI Stent v1 | PCI Stent | 5 | 19 | 5 |

TABLE 5
Escalation Thresholds
SMBG Diabetes - Thresholds

| Range | | | | Exception/ |
|---|---|---|---|---|
| min. | max. | Name | Escalation | Routine |
| no min. | 69 | Low 3 | Quickly escalate to . . . | Exception |
| 70 | 74 | Low 2 | Quickly escalate to . . . | Exception |
| 75 | 79 | Low 1 | Notify Care Circle Member . . . | Exception |
| 80 | 130 | Normal | n/a | n/a |
| 131 | 140 | High 1 | Remind Patient Once | Routine |
| 141 | 160 | High 2 | Notify Care Circle Member . . . | Routine |
| 161 | no | High 3 | [Select Escalation] | [Select type] |
| | max. | | | |

TABLE 6
Missed Parameter Escalation
SMBG Diabetes - Missed Parameter Escalation

| Item No. | Limit | Period | Escalation | Exception/Routine |
|---|---|---|---|---|
| 1 | 9 | times | Notify Care Circle Member 1 | Routine |
| 2 | 12 | times | Notify Care Circle Member 1 | Exception |

1. A protocol management system, comprising a processor, a memory, and communication circuitry, the apparatus being connected to one or more networks via its communication circuitry, the protocol management system further comprising computer-executable instructions stored in the memory of the protocol management system which, when executed by the processor of the protocol management system, cause the protocol management system to perform operations comprising:
archiving a plurality of clinical treatment protocols for various clinical conditions in a standard clinical protocol format, the standard clinical protocol format comprising, for each protocol, a set of elements, the elements comprising a parameter to be tracked, an action to be taken, a symptom to watch for, a period of time for each of the parameter to be tracked, the action to be taken, and the symptom to watch for, indicia of compliance with each element,
escalation path for excursions of parameters outside normal thresholds or to remediate non-compliance of any element, and instructions to the patient in case she experiences a symptom in the defined list;
obtaining a general clinical treatment protocol from a first care provider portal via the one or more networks;
storing the general clinical treatment protocol in the standard clinical protocol format;
providing the general clinical treatment protocol to a second care provider portal via the one or more networks;
receiving, from the second care provider portal, a tailored protocol, the tailored protocol being based on the general clinical treatment protocol and being tailored for a first patient;
providing data regarding the tailored protocol to a first patient portal via the one or more networks;
receiving, from the first patient portal, data regarding compliance with the tailored protocol;
processing the data regarding compliance with the tailored protocol to determine the indicia of compliance with each element of the tailored protocol according to the standard clinical protocol format;
storing the indicia of compliance with each element of the tailored protocol in the standard clinical protocol format; and
allowing an escalation control system to access the indicia of compliance with each element of the tailored protocol in the standard clinical protocol format.

2. An escalation control system, comprising a processor, a memory, and communication circuitry, the apparatus being connected to one or more networks via its communication circuitry, the escalation control system further comprising computer-executable instructions stored in the memory of the protocol management system which, when executed by the processor of the escalation control system, cause the escalation control system to perform operations comprising:
accessing, via a protocol management system, a tailored protocol and an indicia of compliance with one or more elements of a tailored protocol, wherein the tailored protocol and the indicia of compliance with one or more elements of a tailored protocol are stored in the protocol management system in a standard clinical protocol format, the standard clinical protocol format comprising a set of elements, the elements comprising a parameter to be tracked, an action to be taken, a symptom to watch for, a period of time for each of the parameter to be tracked, the action to be taken, and the symptom to watch for, and an indicia of compliance with each element, and wherein the protocol management system stores multiple clinical treatment protocols for various clinical conditions in the standard clinical protocol format;
determining, based at least in part on the indicia of compliance with one or more elements of the tailored protocol, whether to take one or more remedial actions; and
sending, to a patient portal via the one or more networks, a notification of a determined remedial action.

3. The escalation control system of claim 2, wherein determining whether to take one or more remedial actions is based at least in part on an escalation protocol, wherein the escalation protocol comprises data related to a clinical condition and data related to a severity of a non-compliance.

4. The escalation control system of claim 3, wherein the escalation protocol further comprises data related to a combination of clinical conditions.

5. The escalation control system of claim 2, wherein instructions cause the escalation control system to perform further operations comprising, receiving, from an artificial intelligence platform, an adjustment to the escalation protocol.

6. The escalation control system of claim 5, wherein instructions cause the escalation control system to request the adjustment to the escalation protocol.

7. The escalation control system of claim 2, wherein determining whether to take one or more remedial actions is based at least in part on a compliance history of the patient.

8. The escalation control system of claim 2, wherein the determined remedial action is selected for a list comprising: communicating with the patient, communicating with a proxy of the patient, communicating with a care provider, communicating with a proxy of the care provider, scheduling an appointment for the patient, a temporary adjustment to medication dosage, and a temporary adjustment to a therapy.

9. The escalation control system of claim 2, wherein instructions cause the escalation control system to perform further operations comprising sending to the protocol management system a notification to add an element to the tailored protocol.
